(19) United States
(12) Patent Application Publication
(10) Pub. No.: US 2019/0216916 A1
(43) Pub. Date: Jul. 18, 2019

(54) METHODS FOR IMPROVING VACCINE RESPONSIVENESS

(71) Applicant: CHILDREN'S HOSPITAL MEDICAL CENTER, Cincinnati, OH (US)

(72) Inventors: David A. Hildeman, Cincinnati, OH (US); Maha Almanan, Cincinnati, OH (US); Claire A. Chougnet, Cincinnati, OH (US)

(21) Appl. No.: 16/246,966

(22) Filed: Jan. 14, 2019

Related U.S. Application Data
(60) Provisional application No. 62/616,529, filed on Jan. 12, 2018.

(51) Int. Cl. A61K 39/145; A61P 31/16; A61K 47/69; A61K 39/42

(52) U.S. Cl. CPC A61K 39/145; A61P 31/16; A61K 2039/55; A61K 47/6929; A61K 39/42; A61K 47/6913

(57) ABSTRACT

The disclosure provides methods for immunizing a subject in need thereof with a prophylactic vaccine against an infectious disease, the method comprising enhancing the subject's immune responsiveness to the vaccine by administering to the subject an agent that transiently inhibits IL-10 production by follicular helper T (“Tfh”) cells.

### CROSS REFERENCE TO RELATED APPLICATIONS

This application claims the benefit of U.S. Provisional Application No. 62/616,529, filed Jan. 12, 2018, the entire disclosure of which is incorporated herein by reference in its entirety.

### STATEMENT REGARDING FEDERALLY-SPONSORED RESEARCH

This work was supported by funds from the National Institute of Health Grant AG033057.

### FIELD OF THE INVENTION

This disclosure relates to methods for improving vaccine responsiveness in target populations.

### BACKGROUND OF THE INVENTION

Changes in immune function in the elderly result in increased incidence and severity of infections at the same time as a general incidence of decreased responsiveness to prophylactic vaccination (Weinberger, B. Immun. & Ageing 15:3-10 (2018)). Since prophylactic vaccination remains the most effective measure to prevent infections, the national health guidelines of most countries include specific recommendations for vaccinations in the elderly. However, these efforts to reduce infection-related morbidity and mortality in the elderly population are complicated by the concomitant decrease in responsiveness to vaccination that characterizes this group.

Aging is also characterized by a persistent low-grade immune activation which has been implicated in many deleterious processes associated with aging, including Alzheimer's disease, cardiovascular diseases and general frailty. Most inflammatory reactions incite potent anti-inflammatory feedback loops. Interleukin (“IL”) 10 is a broad-ranging and potent anti-inflammatory mediator that has been shown to increase with age in a cross-sectional analysis of more than 465 subjects, ranging in age from 21 to 88 years (Lustig, A. et al., Frontiers Immun. 8:1027 (2017)). A polymorphism in the IL-10 gene (−21082GG) associated with high production of IL-10 in Caucasians is more prevalent in centenarians than in younger individuals (65-73 yrs), and similarly more prevalent in middle-aged controls compared to age-matched patients with myocardial infarction (Lio, D. et al., Genes Immun. 3:30-33 (2002); Lio, D. et al., J. Med. Genet. 41:790-794 (2004)). Elderly men with the highest serum levels of inflammatory cytokines, or with the lowest levels of IL-10, displayed the highest risk of frailty-associated pathologies (Cauley, J. et al., J. Bone Miner. Res. 31: 2129-2138 (2016)). In sum, these studies indicate that IL-10 plays an important role in promoting healthy aging.

However, in cases of persistent or chronic infection by viruses or parasites, IL-10 has been shown to deleteriously suppress the immune response and prevent resolution of infection (Belkaid, Y. et al., J. Exp. Med. 194:1497-1506 (2001); Brooks, D. et al., Nature Med. 12:1301-1309 (2006); Brooks, D. et al., J. Exp. Med. 205:533-541 (2008)). During the acute phase of influenza infection, IL-10 inhibited development of a robust T cell response (McKinstry, K. et al., J. Immunol. 182:7353-7363 (2009)). Earlier studies showed that antibodies against various inflammatory cytokines, including interferon gamma (IFNγ), IL-4, and IL-10 altered the isotypes of the antibodies elicited by an influenza subunit vaccine (Dobber, R. et al., Cell Immunol. 160:185-192 (1995)). The authors noted that the effects of cytokines on isotype switching are dependent on the differentiation stage of B cells, which may differ in young versus aged individuals. Since treatment with anti-cytokine antibodies resulted in increased titers of some types of immunoglobulins while further decreasing other types in aged mice, the authors caution that it is important to know which isotypes are needed to clear a target pathogen in order to potentially improve protection of the elderly against disease by modulating the humoral immune response during vaccination.

IL-10 can be produced by many cells, including those of the innate immune system (notably multiple myeloid cell subsets), the adaptive immune system (T cells and B cells), and even non-immune cells (e.g., keratinocytes and hepatocytes) (Moore, K. et al., Ann. Rev. Immunol. 19:683-765 (2001)). The majority of IL-10 expression in young mice is localized to B cells and CD4+ T cells (CD25+ and CD25−) (Madan, R. et al., J. Immunol. 183:2312-2320 (2009)). In contrast, B cells capable of IL-10 production appear to be decreased in older subjects (van der Geest, K. et al., Exp. Gerontol. 75:24-29 (2016)). The major cellular source of IL-10 production with increased age remains unclear.

Understanding the cellular biology of the age-related increase in IL-10 is critical to understanding how it modulates humoral immunity in the elderly, and realizing the potential for improving vaccine responsiveness in older adults. The present invention addresses these needs.

### SUMMARY OF THE INVENTION

The present invention is based, in part, on the discovery of the specific type of T cell that produces the majority of IL-10 in aged individuals. This discovery enabled the development of methods which target IL-10 production by these cells to enhance vaccine responsiveness in at-risk populations, including the elderly and other immunocompromised populations who may benefit from an enhanced responsiveness to prophylactic vaccination against diseases caused by pathogens.

Accordingly, the disclosure provides methods for immunizing a subject in need thereof with a prophylactic vaccine against an infectious disease and methods of enhancing a subject's immune responsiveness to a prophylactic vaccine. The methods comprise administering to the subject an agent that inhibits IL-10, also referred to herein as “an IL-10 inhibitor.” The disclosure also provides the related use of an agent that inhibits IL-10 in methods for immunization and enhancing immune responsiveness as described herein. In accordance with the methods and uses described here, the agent that inhibits IL-10 may be a direct or indirect inhibitor of IL-10, as described in more detail below. Encompassed are agents that inhibit production of IL-10 by follicular helper T (“Tfh”) cells, including by inhibiting the expression of IL-10 in Tfh cells, as well as agents that inhibit IL-10 signaling, for example by interfering with the binding of IL-10 to its receptor. Accordingly, the IL-10 inhibitor may be selected from a small organic molecule, a peptide, a polypeptide, a polynucleotide, or an antibody, for example an anti-IL-10 receptor antibody. In embodiments, the antibody is a monoclonal antibody, preferably a human or humanized monoclonal antibody that binds to the IL-10 receptor and inhibits or substantially reduces IL-10 binding to its receptor.

In embodiments, the disclosure provides methods for immunizing a subject in need thereof with a prophylactic vaccine against an infectious disease, the method comprising enhancing the subject's immune responsiveness to the vaccine by administering an IL-10 inhibitor to the subject.

In embodiments, the subject in need is an elderly human, a human who has received one or more immunosuppressive agents as part of a therapeutic regimen, for example a chemotherapy regimen or a regimen to prevent rejection in a solid organ transplant recipient, a human who has received one or more regimens of radiation therapy, a human stem-cell transplant recipient, a subject having graft-versus-host disease, a subject having HIV, a subject having end-stage renal disease, a subject having end-stage diabetes, and a subject having end-stage cirrhosis. In embodiments, the subject in need is an elderly human, preferably at least 50 years of age, most preferably at least 65 years of age.

In embodiments, the IL-10 inhibitor is a direct or indirect inhibitor. In embodiments, the IL-10 inhibitor is an agent that inhibits IL-10 production by follicular helper T (“Tfh”). In embodiments, the agent is an inhibitor of IL-10 synthesis in Tfh cells. In embodiments, the IL-10 inhibitor is an agent that inhibits IL-10 binding to its receptor.

In accordance with any of the foregoing embodiments, the IL-10 inhibitor may be a peptide, a polypeptide, a small organic molecule, or an antibody. In embodiments, the IL-10 inhibitor is a monoclonal antibody. In embodiments, the monoclonal antibody is an antibody against IL-10, or the IL-10 receptor, IL-10R.

In accordance with any of the foregoing embodiments, the method may further comprise administering the vaccine to the subject in need thereof.

In accordance with any of the foregoing embodiments, the prophylactic vaccine may be selected from a vaccine against influenza, Streptococcus pneumoniae, tetanus, diphtheria, pertussis, respiratory syncytial virus (RSV), typhoid fever, Japanese encephalitis, yellow fever, Hepatitis A, and Hepatitis B. In embodiments, the prophylactic vaccine is an influenza vaccine.

In embodiments, the vaccine is a Tfh-dependent vaccine.

In an embodiment, the vaccine is a therapeutic vaccine against Herpes zoster. In another embodiment, the vaccine is a therapeutic vaccine against rabies.

In accordance with any of the foregoing embodiments, the IL-10 inhibitor may be administered before, concurrently with, or after the administration of the vaccine. In embodiments, the IL-10 inhibitor is administered substantially at the same time as the vaccine.

In accordance with any of the foregoing embodiments, the subject in need is not one in need of a therapeutic vaccine for the treatment of a chronic infection, or for the treatment of an infection by an organism other than Herpes zoster or rabies. In some embodiments, the subject in need is one who is not already infected with the pathogen targeted by the vaccine at the time the vaccine is administered, with the exception of Herpes zoster or a virus causing rabies.

In accordance with any of the foregoing embodiments, the Tfh cells are defined by the positive cell surface expression of the CD4, CXCR5, and PD1 marker proteins in the absence of FoxP3 expression, i.e., FoxP3⁻ CD4⁺ CXCR5⁺ PD1⁺.

### BRIEF DESCRIPTION OF THE DRAWINGS

FIGS. 1A-B: Aged mice have increased systemic levels of IL-10. (A) Young (white bar, n=6) and old (black bar, n=5) C57BL/6 mice were i.v. injected with biotinylated anti-IL-10 capturing Abs. Serum was collected 24 h later, and IL-10 levels were measured by ELISA. Graph shows the average serum IL-10 (mean±SEM). Data are representative of at least two independent experiments. (B) IL-10 mRNA gene expression was measured by real-time RT-PCR on cDNA isolated from the spleen, liver, gut, lymph nodes, (inguinal, epididymal) white and brown adipose tissue, from individual young (n=4-8) and old (n=5-9) mice. Graph shows the mean fold change in IL-10 mRNA expression calculated by dividing the individual expression level in old mice by the average expression level of the young mice (mean±SEM). Dashed line represents equal level of expression in young and old mice. Data pooled from two independent experiments. *p≤0.05, **p≤0.01, ***p≤0.001, ****p≤0.0001, Student's t-test.

FIGS. 2A-E: CD4+FoxP3− T cells are the major source of IL-10 in aged mice. (A) Splenocytes from young (n=3) and old (n=5) IL-10^{gfp} (Vertex) mice were stained with Abs against CD4, CD8, TCRβ and CD19 and analyzed by flow cytometry. The representative plots show the gating strategy and frequencies of CD4+, CD8+, CD19+ and CD19− that are GFP+. Graphs show the total number (upper) and frequency (middle) of cells that are GFP+ in young (white bar) and old (black bar) mice (mean±SEM). The lower graph shows the level of GFP expression in old CD4+, CD8+, CD19+ and CD19− that are GFP+ (mean±SEM). **p≤0.01, Student's t-test. Data are representative of at least two independent experiments. (B) Splenocytes from young (3 mo, n=4) and old (≥18 mo, n=4) FoxP3-IRES-DTR-GFP mice were enriched for CD4⁺ cells using CD4 T cell isolation kit (Miltenyi) and stained with Abs against CD4, CD62L and CD44. CD4⁺FoxP3^{GFP+}, CD4⁺FOXP3^{GFP-} CD44^{hi} CD62L^{lo} (memory) and CD4⁺FoxP3^{GFP-} CD44^{lo}CD62L^{hi} (naïve) cells were FACS sorted. Purified cells (3×10⁵) were stimulated with PMA and Ionomycin (P+I) for 15 hrs and IL-10 levels in culture supernatants quantified by ELISA. Graph shows the average IL-10 levels (mean±SEM). Data pooled from two independent experiments. (C) Splenocytes from young (n=4) and old (n=4) C57BL/6 mice were stimulated with (P+I), stained with Abs against TCRβ, CD8, Foxp3 and IL-10, and assessed for cytokine production by flow cytometry. The representative plots show the gating strategy and frequencies of Foxp3+ or Foxp3− that are IL-10+ from young or old mice. Graphs show the frequency and the total number of cells that are IL-10+ in young (white bar) and old (black bar) mice (mean±SEM). (D) Young (n=6) and old (n=14) C57BL/6 mice were treated with a single dose (600 μg) of anti-CD4 depleting Ab or isotype control (n=16) at d0. Old Foxp3-DTR C57BL/6 mice (n=6) were treated with a single dose (1.25 μg) of Diphtheria Toxin (DT). Mice were intravenously injected with biotinylated α-IL-10 capturing Ab at d1. Serum was collected at d2 to measure IL-10 levels by ELISA. Graph shows the average serum IL10 levels (mean±SEM) and representative data pooled from two independent experiments. (E) Splenocytes from isotype control old mice (n=8) and old Foxp3DTR C57BL/6 mice treated with a single dose (1.25 μg) of Diphtheria Toxin (DT) (n=6) were stimulated as above (C), stained with Abs against TCRβ, CD8, Foxp3 and IL-10 and analyzed by flow cytometry. Graph shows the frequency of Foxp3− cells that are IL-10+ (mean±SEM). *p≤0.05, **p≤0.01, ***p≤0.001, Student's t-test.

FIG. 3: Splenocytes from young (n=4) and old (n=4) C57BL/6 mice were stimulated with (P+I), stained with Abs against TCRβ, CD8, Foxp3 and IL-10 and analyzed by flow cytometry. Graph shows the mean level of IL-10 in Foxp3+ IL-10+ and Foxp3− IL-10+ cells (mean±SEM). ***p≤0.001, Student's t-test.

FIG. 4: Splenocytes from microbiota-replete conventionally-housed (Con) (n=2-4/group) or germ free (GF) (n=2-5/group) mice were stimulated with (P+I), stained with Abs against TCRβ, CD8, Foxp3 and IL-10 and analyzed by flow cytometry. Graph shows frequency of Foxp3− cells that are IL-10+ (mean±SEM). Data pooled from 3 independent experiments.

FIGS. 5A-C: (A) Splenocytes from young (n=8) and old (n=8) mice were stimulated with (P+I), stained with Abs against TCRβ, CD8, CD49B, LAG3 and IL-10, and analyzed by flow cytometry. The plots and graph show the frequency of indicated subsets in Foxp3− IL-10+ cells (mean±SEM). (B) Splenocytes from young (n=4) and old (n=4) mice were stimulated as above, stained with Abs against TCRβ, CD8, IL-10, IL-17, IL-4 and analyzed by flow cytometry. The plots and graph show the frequency of indicated subsets in Foxp3− cells (mean±SEM). (C) Spleen cells from young (2-mo, n=5) and middle-age (12-mo, n=5) IL-17A^{cre} Rosa26^{YFP} (R26YFP) mice were stimulated as above, stained with Abs against TCRβ, CD8, IL-10, IL-17 and analyzed with flow cytometry. Bar graph shows the (mean±SEM), within the total FOXP3⁻IL-10⁺ cells, the frequency of cells producing IL-17 (IL-17+YFP+, gray), exTh17 (IL-17−YFP+, white) or those that never produced IL-17 (IL-17−YFP−, black).

FIGS. 6A-B: (A) Spleen cells from middle-age (12-mo, n=4) and young (6-weeks, n=3) Foxp3^{Cre}Rosa26^{dTomato} mice were stimulated with (P+I), stained with Abs against TCRβ, CD8, IL-10 and Foxp3, and analyzed with flow cytometry. Plots and bar graph show, within the total Foxp3⁻ IL-10⁺ cells, the frequency of exTreg cells (Foxp3⁻ IL-10^{+dTomato+}). Data pooled from two independent experiments. (B) Splenocytes from young, middle age and old C57BL/6 mice (n≥4/group) were stimulated as above and stained with Ab against TCRβ, CD8, CXCR5, PD1, Foxp3 and IL-10. The representative bar graphs show the frequency of Foxp3− that CXCR5+ PD1+ and those that produce IL-10 (mean±SEM). *p≤0.05, **p≤0.01, Student's t-test.

FIGS. 7A-B: IL-10-producing FoxP3neg CD4+ T cells in aged mice are predominantly Tfh cells. (A) Splenocytes from young (n=6) and old (n=6) C57BL/6 mice were stimulated with (P+I), stained with Abs against TCRβ, CD8, Foxp3, IL-10, IL-21 and analyzed by flow cytometry. The representative histograms and graphs show the frequencies and total numbers of IL-21+ cells within Foxp3− that are IL-10+ (mean±SEM). *p≤0.05, **p≤0.01, Student's t-test. Data are representative of at least two independent experiments. (B) Splenocytes from young (n=4) and old (n=4) C57BL/6 mice were stimulated as above and stained with Abs against TCRβ, CD8, Foxp3, CXCR5, PD-1 and IL-10, and analyzed by flow cytometry. The representative plots and graphs show the frequencies and total numbers of indicated subsets within Foxp3− that are IL-10+ (mean±SEM). *p≤0.05, **p≤0.01, Student's t-test. Data are representative of at least two independent experiments.

FIGS. 8A-B: IL-6 is required for Tfh10 cells and for systemic levels of IL-10 in aged mice. (A) Splenocytes from young and old C57BL/6 or IL-6^{-/-} mice (n≥4/group) were stimulated with (P+I), stained with Ab against TCRβ, CD8, CXCR5, PD1, Foxp3 and IL-10, and analyzed by flow cytometry. The representative plots and bar graphs show the frequency and total number of Foxp3− that CXCR5+ PD1+ and their IL-10 production (mean±SEM). (B) Old C57BL/6 (n=8) and IL-6^{-/-} (n=8) mice were intravenously injected with biotinylated anti-IL-10 Abs, serum was collected 24 hr later, and IL-10 levels were measured by ELISA. Graph shows the average serum IL-10 (mean±SEM). *p≤0.05, **p≤0.01, Student's t-test.

FIG. 9: Old C57BL/6 mice were treated with isotype control (n=6) or α-ICOSL blocking antibody (n=5) on day (0, 3, 6, 9) and sacrificed on day 12. Splenocytes were stimulated with (P+I), stained with Ab against TCRβ, CD4, CD8, CXCR5, PD1, Foxp3 and IL-10, and analyzed by flow cytometry. The representative plots and bar graphs show the frequency of Foxp3− that CXCR5+ PD1+ and the frequency of Foxp3− that IL-10+ (mean±SEM).

FIGS. 10A-C: IL-21 contributes to accrual of Tfh10 cells and regulates the systemic IL-6/IL-10 balance. (A) Splenocytes from young and old C57BL/6 or IL-21^{-/-} mice (n≥4/group) were stimulated with (P+I), stained with Ab against TCRβ, CD8, Foxp3 and IL-21 and analyzed by flow cytometry. Bar graphs show the frequency and total number of Foxp3− that are IL-21+ (mean±SEM). (B) Splenocytes from old C57BL/6 or IL-21^{-/-} mice (n≥3/group) were stimulated as above, stained with Ab against TCRβ, CD8, CXCR5, PD1, Foxp3 and IL-10, and analyzed with flow cytometry. The representative plots and bar graphs show the frequency and total number of Foxp3− CXCR5+ PD1+ and their IL-10 production (mean±SEM). Data are pooled from two independent experiments. (C) Old C57BL/6 (n=4) and old IL-21^{-/-} (n=3) mice were i.v. injected with biotinylated anti-IL-10 and anti-IL-6 capturing Abs, serum was collected 24 h later, and IL-10 and IL-6 levels were measured by ELISA. Graphs show the average serum IL-10 and IL-6 (mean±SEM).

FIG. 11: Old C57BL/6 (19-mo) mice were treated with isotype control (n=6) or α-IL-6 blocking antibody (n=8) on day 0 and sacrificed on day 2. Splenocytes were stimulated with (P+I), stained with Ab against TCRβ, CD8, Foxp3 and IL-10 and analyzed with flow cytometry. The representative bar graph shows the frequency of Foxp3− that are IL-10+ (mean±SEM).

FIGS. 12A-D: IL-21 driven repression of Bim in aged Tfh10 cells results in their enhanced survival. Splenocytes from young (n=4) and old (n=4) mice were stimulated with (P+I), stained with Abs against TCRβ, CD8, Foxp3, IL-10, Ki67, Bim and analyzed by flow cytometry. (A) The graph shows the frequency of Foxp3− IL-10+ cells that are Ki67+(mean±SEM). (B) Graph shows the level of expression of Bim in Foxp3− IL-10+ cells (mean±SEM). (C) Splenocytes from 6-month old wild-type and Bim^{-/-} mice (n=6/group) were stimulated as above, stained with Ab against TCRβ, CD4, Foxp3 and IL-10 and analyzed by flow cytometry. Plots and bar graphs show the frequency and total number of Foxp3− that are IL-10+ (mean±SEM). (D) Splenocytes from C57BL/6 or IL-21^{-/-} mice (n≥3/group) were stimulated as above, stained with Ab against TCRβ, CD8, Foxp3, IL-10 and Bim and analyzed with flow cytometry. Graph shows the level of expression of Bim in Foxp3− CXCR5+ PD1+ that are IL-10+ cells (mean±SEM). *p≤0.05, **p≤0.01, ***p≤0.001, Student's t-test.

FIGS. 13A-C: Tfh10 cells in aged mice manifest diminished levels of BCL6 thereby enabling IL-10 expression. (A) Splenocytes from young and old C57BL/6 mice (n=4/group) were stimulated with (P+I), stained with Ab against, CD8, CXCR5, PD1, Foxp3 and IL-10, and analyzed with flow cytometry. The representative bar graphs show the level of expression of BCL6 and IL-10 in Foxp3− CXCR5+ PD-1+ that are IL-10+ (mean±SEM). (B) Splenocytes from middle age wild-type or CD4^{Cre} BCL6^{f/f} mice (n≥3/group) were stained with Ab against CXCR5, PD1 and Foxp3. The representative plot and bar graph show the frequency of Foxp3-cells that are CXCR5+ PD1+ (mean±SEM). (C) Splenocytes from middle age wild-type or CD4^{Cre}BCL6^{f/f} mice (n≥3/group) were stimulated as above (A), stained with Abs against TCRβ, CD8, Foxp3 and IL-10 and analyzed with flow cytometry. The representative histograms and bar graphs show the frequency and total number of Foxp3-cells that are IL-10+ (mean±SEM). *p≤0.05, **p≤0.01, Student's t-test.

FIGS. 14A-B: IL-10 limits Tfh-dependent vaccine responses in aged mice. (A) Young (n=6) and old (n=5) mice were immunized with NP-KLH in Alum and sacrificed 20 days later. Splenocytes were stained with Abs against CD19, B220, GL7 and Fas and analyzed by flow cytometry. Representative plots identifying GC B cells NP-specific as Fas^{hi}GL7^{hi} that are IgG1+ NP tetramer+. Graphs show the frequency and the total number of splenic B cells that are IgG1+ NP+ (mean±SEM), as well as serum levels of immunoglobulin specific for NP (IgG1) of young vs old mice obtained 20 days after immunization (mean±SEM). (B) Mice were immunized as above and then treated with isotype (n=7) or anti-IL-10R neutralizing Ab (n=8) on day −1, 1, 3, 6, 9 and sacrificed on day 10. Representative plots display the frequency of GC B cells (NP-specific). Graphs show the frequency and the total number of B cells that are IgG1+ NP+ (mean±SEM), as well as serum levels of immunoglobulin specific for NP (IgG1) in mice with or without IL-10R neutralization obtained 10 days after immunization (mean±SEM). *p≤0.05, **p≤0.01, Student's t-test.

FIGS. 15A-C: Tfh10 cells accumulate during aging in humans. Human spleen cells from young (n=8) and old (n=8) individuals were surface stained with Abs against CD3, CD4, CD45RO, CXCR5, PD-1 and Foxp3 and analyzed with flow cytometry. (A) Graph shows the frequency of CD3+ CD4+ CD45RO+ Foxp3− that are CXCR5+ PD-1+ (mean±SEM). (B, C) CD4+ T cells were bead-purified by negative selection, FACS-sorted memory CD4+ T cells (CD45RO+) into Tfh (CD25⁻CD127⁺PD-1⁺CXCR5⁺), Treg (CD25⁺CD127⁻PD-1⁻CXCR5⁻) and other memory cells (CD25⁻CD127⁺PD-1⁻CXCR5⁻). 10,000 cells were stimulated in vitro with anti-CD3/CD28 beads at a 1:1 cell: bead ratio, or unstimulated. After 16 hr, supernatants were collected and analyzed by Luminex. Cytokines were undetectable in unstimulated cultures. Each individual is represented by a symbol, Y (young), O (old) (mean±SEM). ***p≤0.005, Student's t-test.

### DETAILED DESCRIPTION OF THE INVENTION

The present disclosure provides methods for immunizing a subject in need thereof with a prophylactic vaccine against an infectious disease, i.e., a disease caused by a pathogen such as a virus, bacteria, or protozoan. The methods comprise transiently inhibiting IL-10 production in follicular helper T (“Tfh”) cells or otherwise neutralizing IL-10 signaling, thereby enhancing the subject's immune responsiveness to the vaccine. The methods comprise administering to the subject an IL-10 inhibitor. In the context of the present disclosure, Tfh cells are defined by their positive cell surface expression of the cluster of differentiation 4 (“CD4”), C-X-C chemokine receptor type 5 (“CXCR5”), and programmed death 1 (“PD1”) marker proteins in the absence of FoxP3 expression, i.e., FoxP3⁻ CD4+ CXCR5⁺ PDF1⁺. Accordingly, the disclosure also provides methods of inhibiting IL-10 that are targeted to Tfh cells. In embodiments, the methods comprise the use of an IL-10 inhibitor encapsulated in liposome-based nanoparticles targeted to Tfh cells, for example utilitzing a targeting moiety that binds to a cell surface glycoprotein, such as CXCR5. In embodiments, the targeting moiety may be selected from anti-CXCR5 antibodies and the CXCR5 ligand, chemokine (C-X-C motif) ligand 13 (“CXCL13”).

The present methods are directed generally to immunization with a prophylactic vaccine, meaning a vaccine that induces protective immunity against a target pathogen in an individual that is not already infected with the target pathogen at the time the vaccine is administered to the subject. However, in some embodiments, particularly in embodiments where the subject is infected with Herpes zoster or a pathogen causing rabies, the vaccine may be a therapeutic vaccine.

In embodiments of the methods described here, the IL-10 inhibitor may be an agent that inhibits IL-10 directly or indirectly. In embodiments, an IL-10 inhibitor may inhibit production of IL-10 by follicular helper T (“Tfh”) cells. In embodiments, an IL-10 inhibitor may inhibit IL-10 signaling, directly, for example by interfering with the binding of IL-10 to its receptor.

In embodiments, the inhibitor may be a small organic molecule, a peptide, a polypeptide, a polynucleotide, or an antibody, for example an anti-IL-10 antibody. In embodiments, the antibody is a monoclonal antibody, preferably a human or humanized monoclonal antibody that binds to the IL-10 receptor and inhibits or substnatially reduces IL-10 binding to its receptor. In some embodiments, the inhibitor is a polynucleotide, for example an RNA interference-based agent (RNAi) comprised of an RNA complementary to a portion of the mRNA or IL-10 or the IL-10 receptor, optionally further comprising a targeting ligand to direct its delivery to Tfh cells, as described in more detail below.

In embodiments, the IL-10 inhibitor is targeted for delivery to Tfh cells. Targeted delivery systems that may be used include nanoparticles comprised of various materials, for example liposomes, polymers, dendrimers, and magnetic nanoparticles. Nanoparticulate delivery systems suitable for targeting an IL-10 inhibitor to Tfh cells include liposome based nanoparticles such as those described in Gautam et al J. Drug Delivery Sci. Tech. 2017 260-268 and Peer et al. Science 2008 319(5863):627-30. For example, the liposome-based nanoparticles may comprise nanoparticular sized (50-500 nm diameter) liposomes formed from neutral phopholipids comprising a glycosaminoglycan such as hyaluronan to which a targeting moiety is attached. In embodiments the liposomes are loaded with an IL-10 inhibitor selected from a small organic molecule and an RNAi agent, for example an anti-IL-10 siRNA or an IL-10 receptor (“IL-10R”) siRNA. In embodiments, the targeting moiety is selected from an anti-CXCR5 antibody and its ligand CXCL13, in order to effectively target the IL-10 inhibitor loaded liposomes to Tfh cells.

### Antibodies, Peptides, and Polypeptides

In embodiments, the IL-10 inhibitor for use in the methods described here is an antibody, peptide, or polypeptide that binds to the IL-10 receptor and effectively inhibits binding of IL-10 to its receptor. The antibodies for use in the methods described here are preferably monoclonal antibodies, most preferably fully human antibodies, humanized antibodies, camelised antibodies, chimeric antibodies, CDR-grafted antibodies, single-chain Fvs (scFv), disulfide-linked Fvs (sdFv), Fab fragments, F(ab′) fragments, or antigen-binding fragments of any of the foregoing. The antigen-binding fragments are fragments of the immunoglobulin molecules that contain an IL-10 receptor binding site. Fab, Fab′, F(ab′)2 and Fv fragments lack the heavy chain constant fragment (Fc) of an intact antibody and may be preferable over an intact antibody due to their rapid clearance from the systemic circulation and fewer off-target effects. Such fragments are produced from intact antibodies using methods well known in the art, for example by proteolytic cleavage with enzymes such as papain (to produce Fab fragments) or pepsin (to produce F(ab′)2 fragments). In embodiments, the antigen-binding fragment is a dimer of heavy chains (a camelised antibody), a single-chain Fvs (scFv), a disulfide-linked Fvs (sdFv), a Fab fragment, or a F(ab′) fragment. Such fragments may also be fused to another immunoglobulin domain including, but not limited to, an Fc region or fragment thereof. The skilled person will appreciate that other fusion products may be generated, including but not limited to, scFv-Fc fusions, variable region (e.g., VL and VH)-Fc fusions, and scFv-scFv-Fc fusions. Immunoglobulin molecules can be of any type, including, IgG, IgE, IgM, IgD, IgA and IgY, and of any class, including IgG1, IgG2, IgG3, IgG4, IgA1 and IgA2), or of any subclass.

As noted above, the antibodies for use in the methods described here are preferably monoclonal antibodies. A monoclonal antibody is derived from a substantially homogeneous population of antibodies specific to a particular antigen, which population contains substantially similar epitope binding sites. Such antibodies may be of any immunoglobulin class including IgG, IgM, IgE, IgA, and any subclass thereof. Methods for monoclonal antibody production are well known in the art. Preferably, a monoclonal antibody for use in the methods and compositions of the invention is produced using hybridoma technology.

A human antibody is one in which all of the sequences arise from human genes. Human antibodies include antibodies having the amino acid sequence of a human immunoglobulin and include antibodies isolated from human immunoglobulin libraries or from mice that express antibodies from human genes.

A humanized antibody is one which comprises a framework region having substantially the same amino acid sequence as a human receptor immunoglobulin and a complementarity determining region (“CDR”) having substantially the same amino acid sequence as a non-human donor immunoglobulin. A humanized antibody comprises substantially all of at least one, and typically two, variable domains (Fab, Fab′, F(ab′)2, Fv) in which all or substantially all of the CDR regions correspond to those of the non-human donor immunoglobulin (i.e., the donor antibody) and all or substantially all of the framework regions of the human acceptor immunoglobulin. A humanized antibody can be produced using variety of techniques known in the art, including but not limited to, CDR-grafting, veneering or resurfacing, chain shuffling.

A chimeric antibody comprises non-human variable region sequences and human constant region sequences. A chimeric antibody may be monovalent, divalent or polyvalent. A monovalent chimeric antibody is a dimer formed by a chimeric heavy chain associated through disulfide bridges with a chimeric light chain. A divalent chimeric antibody is a tetramer formed by two heavy-light chain dimers associated through at least one disulfide bridge. A polyvalent chimeric antibody can also be produced, for example, by employing a heavy chain constant region that aggregates (e.g., from an IgM heavy chain).

A “camelised” antibody is one having a functional antigen binding site comprising only the heavy chain variable domains (VH), rather than the conventional antigen binding site which comprises both the heavy and the light chain variable domains (VL). Preferably, a camelised antibody comprises one or two VH domains and no VL domains. Preferably, a camelised antibody comprises two VH domains. Methods for making camelised antibodies are known in the art.

The antibodies for use in the methods and compositions of the invention may be produced by recombinant expression using techniques known in the art.

### RNA Based Inhibitors

In some embodiments, the IL-10 inhibitor one is one that inhibits the production of IL-10 by a cell, for example by decreasing expression of the IL-10 gene in the cell, preferably in Tfh cells. In embodiments, the inhibitor is a polynucleotide, preferably a single or double stranded ribonucleic acid (RNA) agent. An RNA agent inhibits expression of a target gene, for example, by catalyzing the post-transcriptional cleavage of the target mRNA, or by inhibiting transcription or translation of the target mRNA. In accordance with some embodiments, the RNA agent is targeted to inhibit expression of the IL-10 gene or the IL-10 receptor gene. In embodiments, the inhibitor is a double stranded or single stranded RNA interference-based agent (RNAi). The RNAi agent may be based on a microRNA (miRNA), a short hairpin RNA (shRNA), or a small interfering RNA (siRNA) that may be single or double stranded. The RNAi agent comprises a region that is at least partially, and in some embodiments fully, complementary to the target RNA. Although perfect complementarity is not required, the correspondence should be sufficient to enable the RNAi agent, or its cleavage product in the case of double stranded siRNA or RNAi agents comprising cleavable linkers, to direct sequence specific silencing of the target mRNA, e.g., by RNAi-directed cleavage of the target mRNA. Over 20 RNAi-based therapeutic agents are in clinical trials in the United States and this technique has shown considerable promise in selectively inhibiting target gene expression to achieve clinical results. See e.g., Bobbin and Rossi Annu Rev. Pharmacol Toxicol (2016) 56:103-122. In embodiments, the RNAi agent may further comprise a delivery system, for example a liposomal or nanoparticle-based delivery system.

In embodiments, the RNAi agent further comprises one or more modified nucleotides, particularly of the single stranded regions of a double-stranded RNA or the terminal regions of a single stranded RNA. In the case of a double-stranded RNA, the dsRNAi agent typically includes at least one 3′ overhang of about 2-5 nucleotides and may include one or two 5′ or 3′ overhangs, which can be the result of one strand being longer than the other, or of two strands of the same length being staggered. Modifications may include those that stabilize the 3′ and/or 5′ ends of the RNAi agent against the activity of exonucleases, for example modifications of the 2′ hydroxy (OH) group of the ribose sugar to a 2′ fluorine or 2′ hydyroxymethyl moiety. Other modifications may include the use of deoxyribonucleotides, e.g., deoxythymidine, instead of ribonucleotides at the 2′ OH group of the ribose sugar, and modifications in the phosphate group, e.g., phosphothioate modifications.

In some embodiments, the RNAi agent further comprises a targeting moiety. The targeting moiety may optionally be conjugated to the RNAi agent, optionally via a linker, or alternatively the targeting moiety may be conjugated to a delivery vehicle, such as a liposome-based nanoparticle. In embodiments, the targeting moiety targets delivery of the RNAi agent to Tfh cells. In embodiments, the targeting moiety comprises a C-X-C chemokine receptor type 5 (“CXCR5”) ligand. In embodiments, the CXCR5 ligand is the chemokine (C-X-C motif) ligand 13 (“CXCL13”), or a CXCR5 binding fragment thereof. In embodiments, the targeting moiety is an anti-CXCR5 antibody.

In embodiments, the RNAi agent is an siRNA targeted to IL-10 mRNA or IL-10R mRNA in a human Tfh cell, the siRNA being encapsulated in a liposome-based nanoparticle ranging in size from about 50-500 nanometers (“nm”) mean diameter, preferably about 50-100 nm mean diameter, the liposomes formed from neutral phopholipids comprising a glycosaminoglycan, preferably hyaluronan, to which a targeting moiety is attached. In embodiments, the targeting moiety is selected from an anti-CXCR5 antibody and its ligand CXCL13, which may be covalently attached to the glycosaminoglycan component of the liposome, thereby targeting the anti-IL-10 or anti-IL-10R siRNA loaded liposomes to human Tfh cells.

In some embodiments, the IL-10 inhibitor is a small organic molecule. In this context, the term “small organic molecule” refers to organic compounds having a molecular weight of less than about 5,000 grams per mole, less than about 1,000 grams per mole, less than about 500 grams per mole, or less than about 100 grams per mole, and salts, esters, and other pharmaceutically acceptable forms of such compounds. In embodiments, the molecular weight of a small organic molecule of the disclosure is between 100 and 500 grams per mole, or between 500 and 1,000 grams per mole, or between 1,000 and 5,000 grams per mole. In embodiments, the small organic molecule is encapsulated within a liposome-based nanoparticle delivery system targeted for delivery to Tfh cells, the liposome-based nanoparticle ranging in size from about 50-500 nanometers (“nm”) mean diameter, preferably about 50-100 nm mean diameter, and formed from neutral phopholipids comprising a glycosaminoglycan, preferably hyaluronan, to which a targeting moiety is attached. In embodiments, the targeting moiety is selected from an anti-CXCR5 antibody and its ligand CXCL13, which may be covalently attached to the glcosaminoglycan component of the liposome, thereby targeting the anti-IL-10 or anti-IL-10R siRNA loaded liposomes to human Tfh cells.

In embodiments of the methods described here, the IL-10 inhibitor may be administered before, concurrently with, or after the administration of the vaccine. In embodiments, the IL-10 inhibitor is administered substantially at the same time as the vaccine. In this context, “substantially at the same time” means either concurrently with at the same time or within a few minutes, for example within 1-15 minutes of each other. In other embodiments, the IL-10 inhibitor is administered before the prophylactic vaccine, for example at least 1 hour before. In some embodiments, the IL-10 inhibitor is administered 12 hours or 24 hours before the vaccine. In other embodiments the IL-10 inhibitor is administered after the prophylactic vaccine, for example at least 1 hour after. In some embodiments, the IL-10 inhibitor is administered 12 hours or 24 hours after the vaccine.

In embodiments, the subject in need is a human subject. In embodiments, the subject in need is an elderly human, a human who has received one or more immunosuppressive agents as part of a therapeutic regimen, for example a chemotherapy regimen or a regimen to prevent rejection in a solid organ transplant recipient, a human who has received one or more regimens of radiation therapy, a human stem-cell transplant recipient, a subject having graft-versus-host disease, a subject having HIV, a subject having end-stage renal disease, a subject having end-stage diabetes, and a subject having end-stage cirrhosis.

In embodiments, the subject in need is an elderly human. In the context of the present disclosure an elderly human is one who is at least 50 years of age or older, preferably at least 65 years of age or older.

In some embodiments the subject may be a non-human mammal, for example a dog, a cat, a pig, a horse, a cow, or a rabbit.

In embodiments, the methods may further comprise administering the vaccine to the subject in need thereof, either before, concurrently with, or after, the administration of the agent that inhibits IL-10 production by Tfh cells. Administration of the vaccine may be by any suitable route of administration, for example intramuscular, subcutaneous, intranasal, pulmonary, and oral. In embodiments, administration of the prophylactic vaccine is by intramuscular or subcutaneous injection. In embodiments, administration of the prophylactic vaccine is intranasally.

A vaccine administered to a subject in need thereof according to the methods described here may be a live attenuated vaccine, an inactivated vaccine, e.g., one in which the pathogen of the vaccine is killed or destroyed by chemical treatment, heat, or radiation, a subunit vaccine, or a conjugate vaccine. In embodiments, the prophylactic vaccine may be a DNA vaccine, an RNA vaccine, or a vaccine comprising virus-like particles (VLPs).

In embodiments, the vaccine administered to a subject in need thereof according to the methods described here is a vaccine against an infectious disease-causing organism, for example a virus, bacterium, or protozoan. In embodiments, the vaccine is a vaccine against a virus. In embodiments, the virus is selected from influenza. In embodiments, the vaccine is a vaccine against an influenza virus, including but not limited to, a vaccine targeting a plurality of influenza viruses such as influenza A H1N1, influenza A H3N2, and influenza B. In embodiments, the prophylactic vaccine is a trivalent or quadrivalent influenza vaccine, for example, a vaccine marketed under the tradename Fluzone® (Sanofi) or Fluad™. In embodiments, the vaccine is a vaccine against a bacterial pathogen. In embodiments, the bacterium is selected from Streptococcus pneumoniae, Clostridium tetani, Corynebacterium diptheriae and Bortadella pertussis. In embodiments, the vaccine is a pneumococcal vaccine. In embodiments, the pneumococcal vaccine is a conjugated vaccine, e.g., one covering 7, 10 or 13 serotypes such as sold under the tradename Prevnar13® (Pfizer), or a polysaccharide vaccine, e.g., one containing 23 serotypes such as sold under the tradename Pneumovax® (Merck). In embodiments, the vaccine is a vaccine against respiratory syncytial virus (RSV). In embodiments, the vaccine is selected from a vaccine against typhoid fever, Japanese encephalitis, yellow fever, Hepatitis A and Hepatitis B.

In some embodiments, the vaccine is a therapeutic vaccine directed against Herpes zoster or rabies. In embodiments, the therapeutic vaccine is a vaccine against Herpes zoster, for example as sold under the tradename Zostavax® (Merck) or Shingrix® (GlaxoSmithkline).

In embodiments, the vaccine administered to a subject in need thereof according to the methods described here is a vaccine against a nosocomial pathogen. In embodiments, the nosocomial pathogen is selected from the group consisting of Clostridium difficile, Staphlococcus aureus, Klebsiella pneumonia, Escherichia coli and Salmonella spp. In embodiments, the nosocomial pathogen is selected from C. difficile and S. aureus.

In embodiments, the methods may further comprise administration of an adjuvant to the subject in need thereof, either before, concurrently with, or after, the administration of the agent that inhibits IL-10 production by Tfh cells. The methods may optionally further comprise administration of the adjuvant, before, concurrently with, or after, the administration of the vaccine. In some embodiments, the vaccine may be formulated with an adjuvant. Exemplary adjuvants that may be used in accordance with the methods described here include alum and its aluminum salts. In embodiments, the adjuvant may be selected from the group consisting of alum, aluminum hydroxide, aluminum phosphate, and similar aluminum salts. Other adjuvants may also be used. In embodiments, the adjuvant may be selected from the group consisting of a lipopolysaccharide- (LPS) derived adjuvant, for example 3-deacyl-monophosphoryl lipid A, also referred to simply as “monophosphoryl lipid A” or “MPL”, which is sold under the tradename AS04™ (GlaxoSmithKline), a squalene based adjuvant, for example, MF59® (Novartis) or AS03™ (GlaxoSmithKline), a saponin-based adjuvant (e.g., ISCOMs), and a Freund's adjuvant.

### EXAMPLES

Immune responses deteriorate with age and result in the decline of vaccine responsiveness. Chronic low-level inflammation termed inflammaging may underlie the impairment of adaptive immune responses; however, the underlying mechanisms remain unclear. Here, we show that aged mice exhibit increased systemic IL-10 that is primarily produced by FoxP3− CD4+T cells. Further, flow cytometric analysis revealed that the majority of these cells manifest a T follicular helper cell (Tfh) profile, which we are referring to as Tfh10 cells. Intriguingly, Tfh10 cells express lower levels of BCL6 thereby enabling IL-10 expression. Importantly, neutralization of IL-10R signaling significantly restores Tfh-dependent antibody responses in aged mice. Finally, IL-6 and IL-21 are required for the accumulation of Tfh10 cells with IL-21 promoting Tfh10 survival sufficient to maintain a systemic balance between IL-6 and IL-10. We propose that Tfh10 cells counter-regulate inflammaging but, in so doing, lead to impaired humoral responses with age. Our data show that systemic levels of IL-10 are increased in aged mice and negatively impact vaccine responsiveness. Notably, we found that CD4+ FoxP3−, not classic FoxP3+, cells were required for increased systemic IL-10 levels in aging. Further, these IL-10-producing T cells bore markers of T follicular helper cells (Tfh), were present in both mice and humans, and required IL-6 for their accumulation. Interestingly, IL-21, another promoter of Tfh homeostasis, was also required for the accrual of these cells, and, importantly, to regulate the systemic balance between IL-6 and IL-10. Mechanistically, we also found that the canonical Tfh transcription factor, BCL6, was downregulated with age in Tfh cells, permitting their IL-10 production. Together, our data show that inflammation and anti-inflammation are linked via IL-21 production, which promotes accrual of IL-10-secreting Tfh (Tfh10) cells that function to dampen both immune responsiveness and IL-6-driven inflammaging.

Aged Mice have Increased Systemic Levels of IL-10.

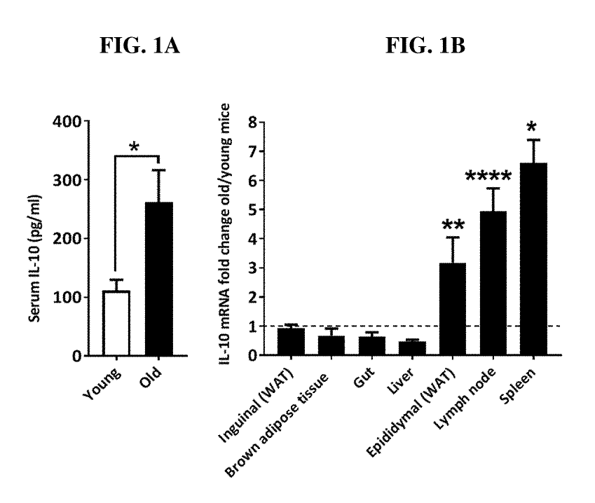

While IL-10 levels have been shown to increase in aged humans (Lustig, A. et al., Frontiers Immun. 8:1027 (2017)), it is unclear if IL-10 levels increase in aged mice. Using the sensitive in vivo cytokine capture assay (IVCCA) we found that steady-state levels of IL-10 in the serum were increased 2-3 fold in old compared to young mice (FIG. 1A). To determine the potential sources of this enhanced IL-10, we examined various lymphoid and non-lymphoid tissues and found an increase in IL-10 mRNA in the epididymal white adipose tissue (WAT), lymph nodes, and spleen of aged, compared to young mice (FIG. 1B). These data show that the systemic levels of IL-10 are increased with age and that secondary lymphoid organs appear to be major contributors of augmented IL-10 expression in aging.

### CD4+FoxP3− T Cells are the Major Source of IL-10 in Aged Mice.

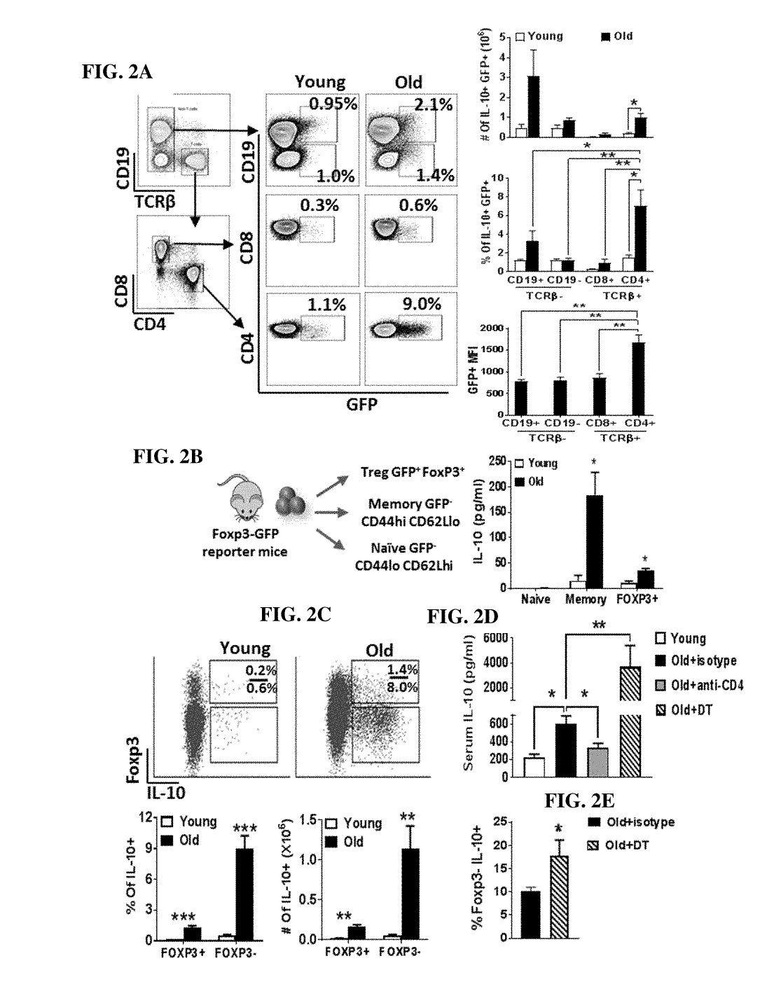

To identify cells with enhanced IL-10 production in aged mice, we took advantage of IL-10-reporter (VertX) mice, which possess an IL-10-IRES-eGFP cassette in the endogenous IL-10 locus (Madan, R. et al., J. Immunol. 183:2312-2320 (2009)). VertX mice allowed us to examine baseline IL-10-production directly ex vivo, in the absence of exogenous stimulation, as GFP levels in these mice directly correlate with IL-10-production (Madan, R. et al., J. Immunol. 183:2312-2320 (2009)). Flow cytometric analysis of spleen cells in aged versus young VertX mice revealed a significantly increased frequency of GFP+ (IL-10+) cells in multiple cell types, but the largest increase was observed in CD4+ T cells (FIG. 2A). As B cells are the predominant immune cell type in the spleen, the total numbers of GFP+ B cells were increased with age. However, there was no significant difference in the numbers of aged IL-10+ B cells compared to IL-10+ CD4+ T cells (FIG. 2A). Instead, the largest increase in the frequency of IL-10-producing cells was in CD4+ T cells (FIG. 2A). In addition, the level of IL-10 produced per cell was significantly higher in CD4+ T cells than in either CD8+ T cells, CD19− or CD19+ B cells (FIG. 2A).

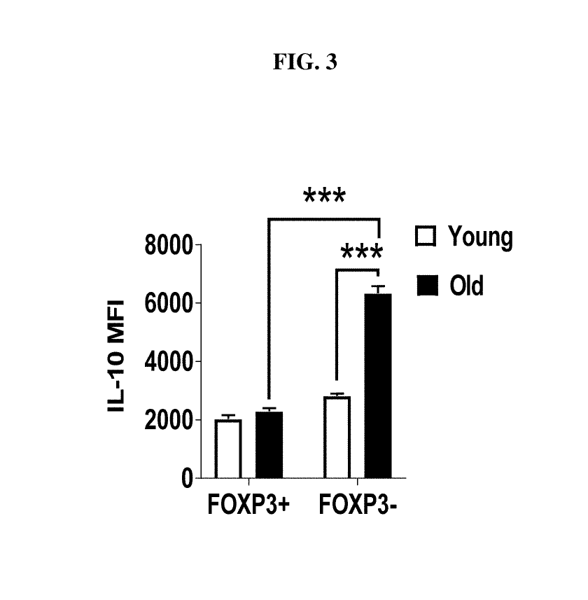

Because FoxP3+ regulatory T cells (Treg) are a well-known source of IL-10 in young mice, and their frequency is increased in old mice (Raynor, J. et al., Curr. Opin. Immunol. 24:482-487 (2012)), we next determined whether they were the major contributor to this increased IL-10 in aged mice. Staining for FoxP3 in VertX mice while maintaining GFP expression is technically infeasible, so we sorted naive (CD4⁺CD44^{lo}CD62L^{hi}FoxP3GFP^{neg}), memory (CD4⁺CD44^{hi}CD62L^{lo}FoxP3GFP^{neg}) and regulatory (FoxP3GFP^{pos}) T cells from young and aged FoxP3-DTR-GFP mice (Kim, J. et al., Nature Immunol. 8:191-197 (2007)), stimulated them with PMA and Ionomycin (P+I), and measured their production of IL-10 by ELISA. As expected, naïve T cells produced little IL-10 whether they were from young or old mice (FIG. 2B). IL-10 production from FoxP3+ Treg was slightly increased in aged mice (˜2-fold), (FIG. 2B). However, IL-10 production from aged FoxP3− memory T cells was increased >10-fold, (FIG. 2B). Similarly, flow cytometric analysis of spleen cells of WT mice showed that the frequency of IL-10-producing CD4+ FoxP3+ cells was increased slightly with age, while IL-10-producing FoxP3− CD4+ T cells were ˜10-times more frequent with age (FIG. 2C). Again, Foxp3− CD4 T cells showed significantly higher expression of IL-10 per cell compared to their young counterparts and aged Foxp3+ cells (FIG. 3).

Together, these three independent approaches show that CD4+ FoxP3− cells have the highest capacity for IL-10-production in the spleens of aged mice. In addition, they are required for the increased systemic levels of IL-10, as depletion of >95% of CD4+ T cells in the spleens of old mice nearly returned the serum levels of IL-10 to levels observed in young mice (FIG. 2D). In contrast, depletion of FoxP3+ T cells increased systemic IL-10 levels and the frequency of IL-10-producing CD4+ T cells (FIG. 2E). Thus, FoxP3−, but not FoxP3+, CD4+ T cells are required for the increased systemic levels of IL-10.

### Accrual of IL-10-Producing CD4+FoxP3− T Cells Occurs in Germ-Free Animals.

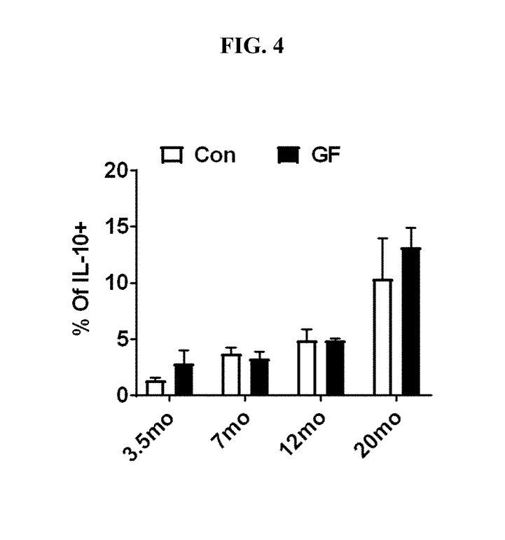

Recent work has shown that the microbiome changes with age (Odamaki et al., 2016). Further, alterations in the microbiome can affect IL-10 production from CD4+ FoxP3+ and FoxP3− T cells (Mazmanian, S. et al., Nature 453:620-625 (2008); Round, J. et al., PNAS (USA) 107:12204-12209 (2010)). To test whether the microbiome affects the accumulation of IL-10-producing cells, we aged several cohorts of mice in a germ-free facility. Interestingly, the accumulation of IL-10+ CD4+ FoxP3− cells was similar between age-matched mice housed under specific pathogen free conditions and germ-free animals across a range of ages (FIG. 4). Interestingly, we also show that age-driven changes to the microbiome (Thevaranjan, N. et al., Cell Host Microbe 21:455-466 (2017)) do not appear to alter the accrual of IL-10-producing cells as these cells accumulate in germ-free mice. Further, the age-driven accrual of IL-10-producing cells occurred at four different institutions including: Cincinnati Children's Hospital, Indiana University/Purdue University Indianapolis, the University of Alabama-Birmingham, and the Research Center Borstel in Germany. It is unlikely that the microbiomes of mice are the same at these different institutions. Therefore, the microbiome is not required for the accumulation of IL-10-producing CD4+ FoxP3− T cells.

### IL-10-Producing CD4+FoxP3− T Cells in Aged Mice are Predominantly Tfh Cells.

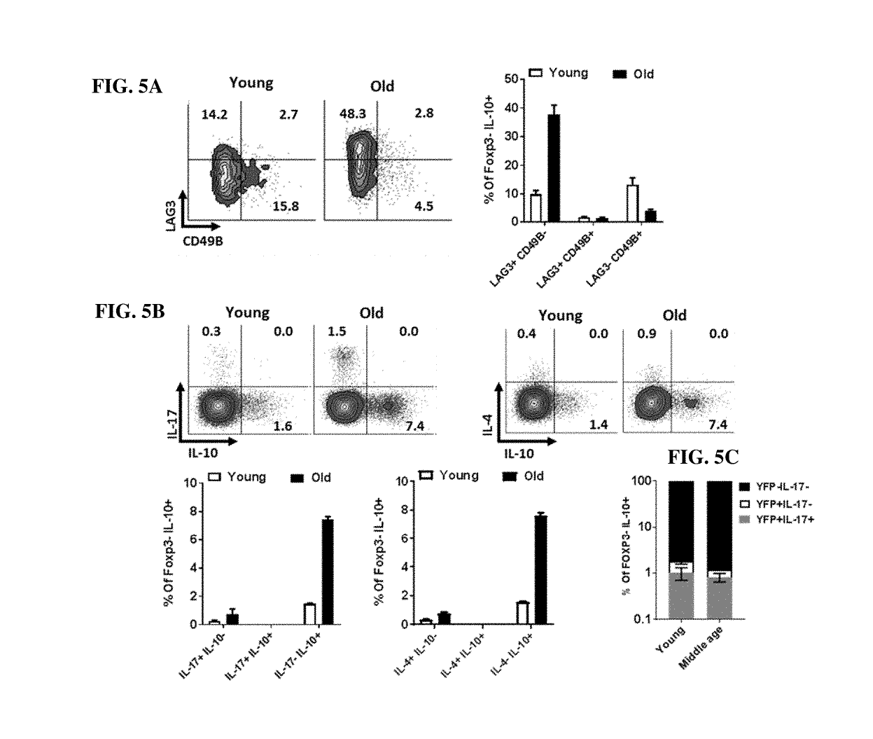

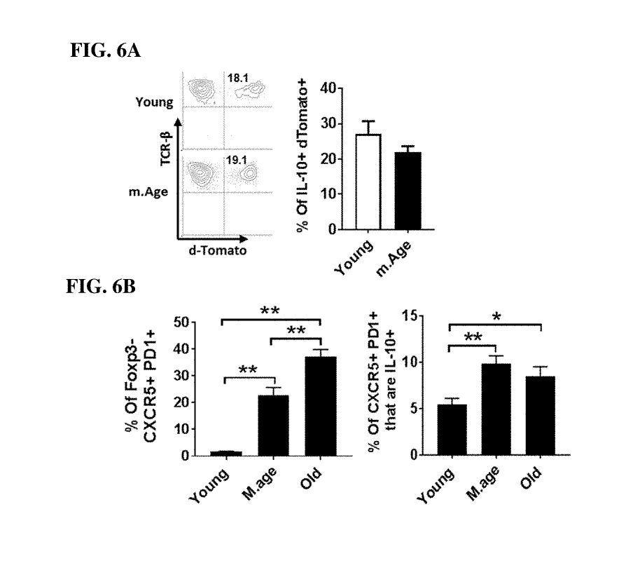

Several distinct subsets of FoxP3− CD4+ T cells have been reported to produce IL-10, predominantly Th2, type I regulatory (TR1) T cells, “exTh17” cells, and exTreg cells (Gagliani, N. et al., Nature 523:221-225 (2015); Roncarolo, M. et al., Curr. Topics Microbiol. Immunol. 380:39-68 (2014); Wang, Z. et al., J. Immunol. 174:2098-2105 (2005)). Although they expressed LAG3, it is unlikely that the majority of the IL-10-producing cells were TR1 cells as they lacked expression of CD49b (FIG. 5A), an important marker on TR1 cells (Gagliani, N. et al., Nature Med. 19:739-746 (2013)). Very few IL-10+ CD4+T cells were capable of IL-4 or IL-17A co-production, ruling out the possibility that these were Th2 or Th17 cells (FIG. 5B). Next, analysis of IL-17A fate tracking mice (Hirota, K. et al., Nat. Immunol. 12:255-263 (2011) revealed that the frequency of “exTh17” cells within IL-10+ FoxP3− CD4+ T cells from aged mice was ˜1% (FIG. 5C). Analysis of exTreg cells using FoxP3-CreRosa^{loxstoplox} dTomato mice (Zhou, X. et al., J. Exp. Med. 205:1983-1991 (2008); Madisen, L. et al., Nat. Neurosci. 13:133-140 (2010)) revealed that ˜20% of the IL-10+CD4+ T cells were dTomato+ GFP− “exTregs” in both young and old mice (FIG. 6A). Thus, neither Th2, TR1, exTh17, nor exTreg make up the bulk of the IL-10-producing CD4+ T cells that accumulate in aged mice.

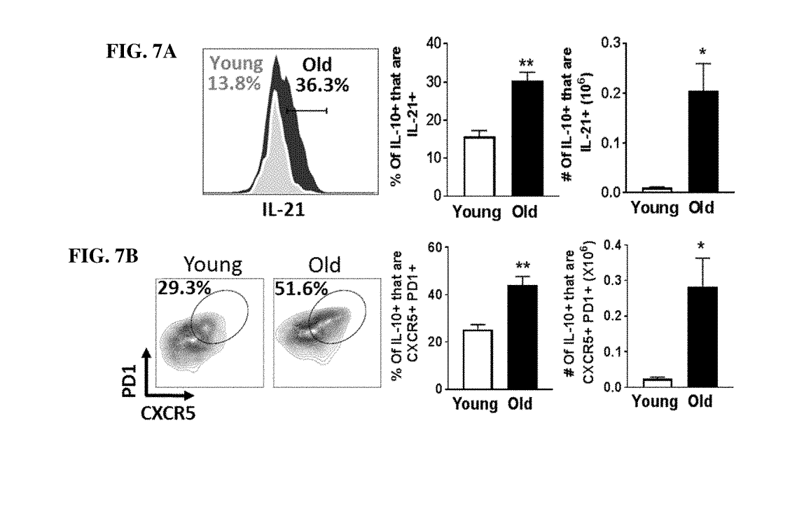

In our investigation of cytokine co-production by IL-10-producing CD4+ T cells, we found that the frequency and total numbers of IL-10+ cells that co-produced IL-21 was significantly increased in aged, compared to young, mice (FIG. 7A). As IL-21 is typically produced by T follicular helper (Tfh) cells, we next assessed the frequency of IL-10+ CD4+ FoxP3− T cells that expressed CXCR5 and PD1, two canonical surface markers of Tfh cells, in conjunction with the transcription factor BCL6 (Haynes, N. et al., J. Immunol. 179:5099-5108 (2007); Johnston, R. et al., Science 325:1006-1010 (2009)). Strikingly, we found that the majority of IL-10+ FoxP3− CD4+ T cells were CXCR5+ and PD1+ in old mice (FIG. 7B). Further, there was a progressive age-related accrual of CXCR5+ PD1+ Tfh cells, including those that produce IL-10 (FIG. 6B). Thus, the majority of the IL-10-producing T cells that accumulate with age bore markers of Tfh cells so for clarity, we will refer to them as Tfh10 cells.

### IL-6 is Required for Tfh10 Generation and Systemic Increase of IL-10 in Aged Mice.

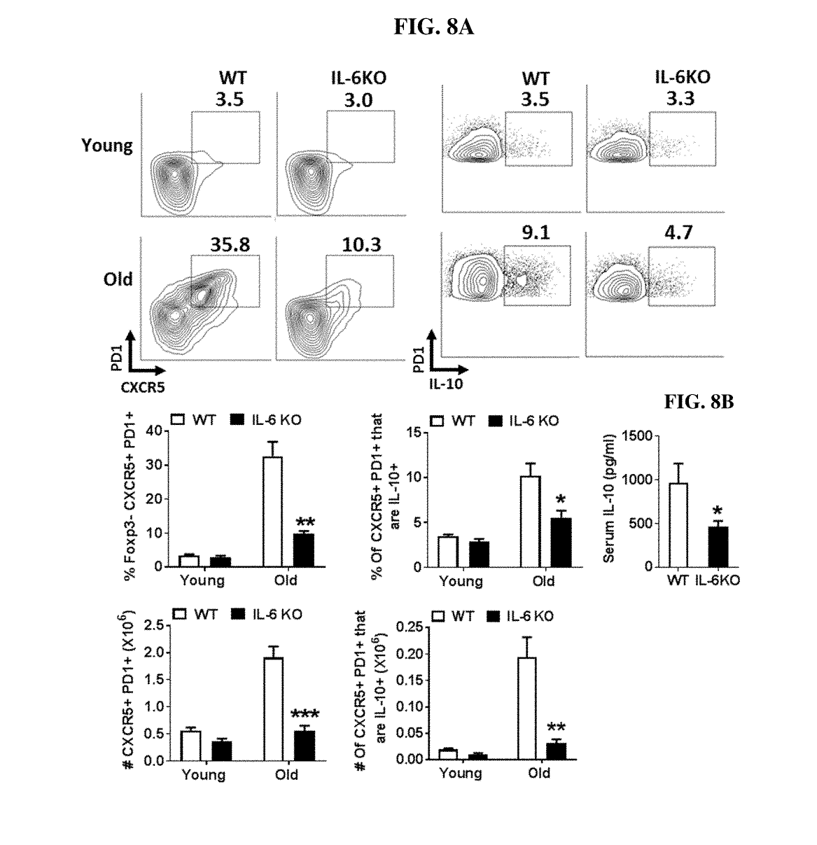

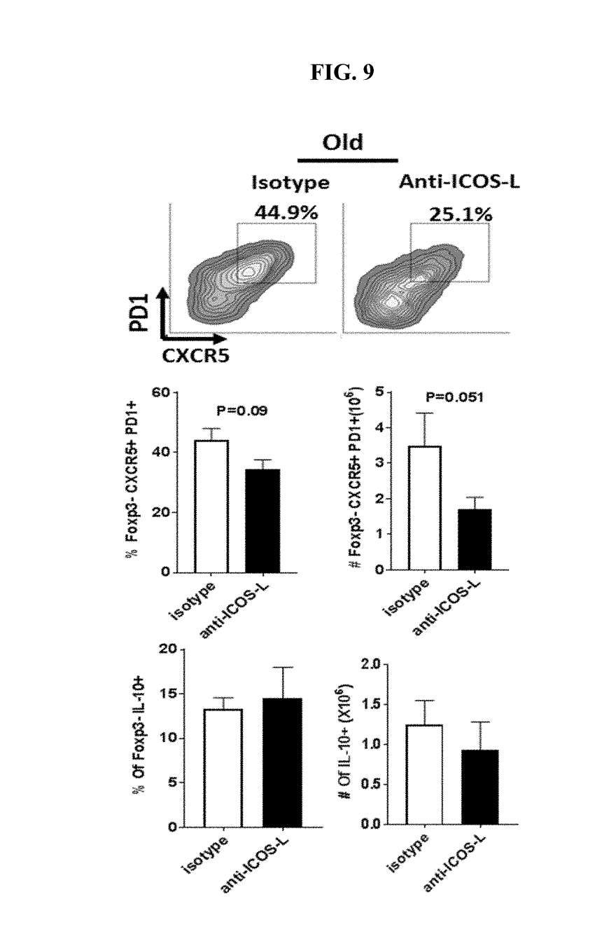

We next examined the role of IL-6 in the accrual of Tfh10 T cells because IL-6: (i) controls Tfh development; (ii) promotes IL-10-production from CD4+ T cells; and (iii) is a key inflammatory cytokine that is increased with age. To determine whether IL-6 promotes the accrual of Tfh10 cells with age, we aged IL-6^{-/-} mice ≥16 mo and examined the proportion of Tfh10 cells. While no difference in the Tfh cells (including those that produce IL-10) was observed between young WT and IL-6^{-/-} mice, aged IL-6^{-/-} mice exhibited a dramatic reduction in the frequency of Tfh10 cells compared to aged WT mice (FIG. 8A). Consistent with Tfh10 T cells being a major source of IL-10 in vivo, we found that systemic levels of IL-10 were significantly decreased in aged IL-6^{-/-} mice (FIG. 8B). To determine whether IL-6 was required for the development or survival of IL-10-producing FoxP3−CD4+ T cells, we blocked IL-6 after Tfh cells were formed and found that neutralization of IL-6 did not reduce the frequency or numbers of IL-10-producing cells (FIG. 9). Thus, IL-6 is required for the accrual of Tfh10 cells, likely by promoting their initial development.

### IL-21 Promotes Accumulation of Tfh10 Cells and Regulates Systemic IL-6/IL-10 Balance.

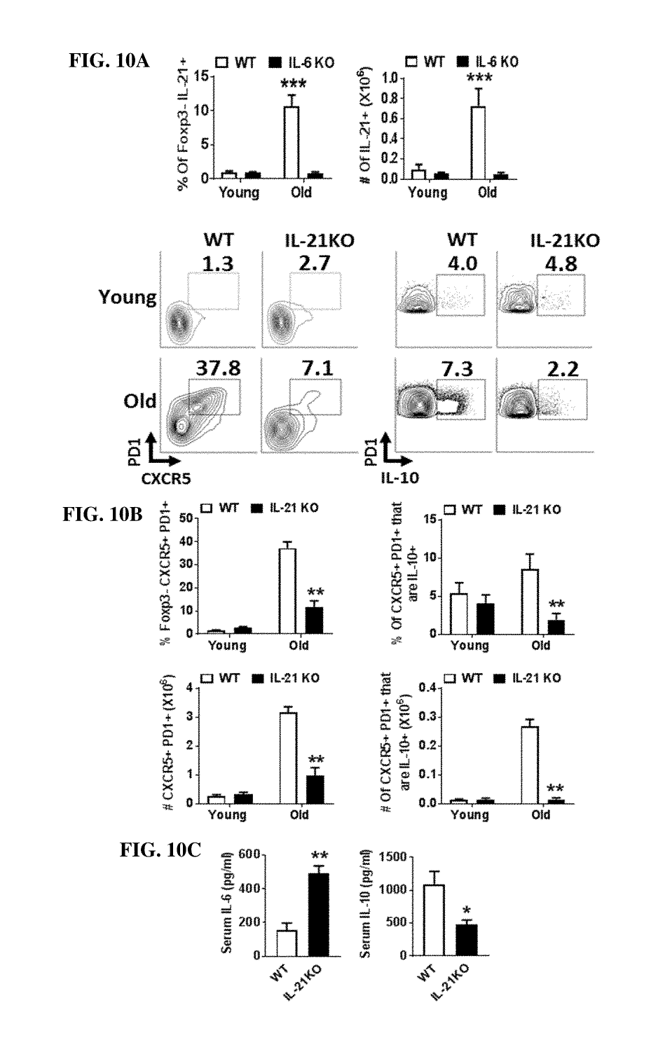

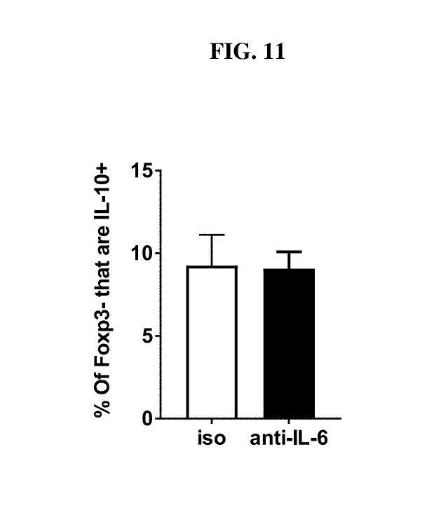

As IL-21 is a critical cytokine produced by Tfh cells (Nurieva, R. et al., Nature 448:480-483 (2007), we next examined whether IL-6 promoted IL-21 production by CD4+ T cells. As expected, and consistent with elevated Tfh cells with age, the proportion and absolute number of IL-21+ CD4+ T cells was significantly increased in aged, compared to young, mice (FIG. 10A). Notably, in the absence of IL-6, the frequency and total numbers of IL-21-producing CD4+ T cells was completely abrogated (FIG. 10A). As IL-21 is also critical for the development and homeostasis of Tfh cells (Vogelzang, A. et al., Immunity 29:127-137 (2008), we reasoned that IL-21 could contribute to the accrual of Tfh10 T cells with age. Similar to aged IL-6^{-/-} mice, the loss of IL-21 prevented age-driven accrual of Tfh cells including those that produce IL-10 (FIG. 10B). Again, consistent with the loss of Tfh10 T cells, levels of systemic IL-10 were reduced in aged IL-21^{-/-} mice compared to aged wild type controls (FIG. 10C). Strikingly, the levels of IL-6 were increased in IL-21-deficient aged mice (FIG. 10C). Together, these data show that IL-21 is critical to balance systemic inflammation (e.g IL-6/IL-10 levels), likely by promoting the accrual of Tfh10 cells. As IL-6 and IL-21 have been reported to increase ICOS which is critical for survival of Tfh cells (Akiba, H. et al., J. Immunol. 175:2340-2348 (2005)), we considered the possibility that increased levels of ICOS on aged Tfh cells could be contributing to their accumulation. Interestingly, we found a significant but marginal effect of ICOS-L neutralization on overall Tfh cell number and no effect on IL-10 producing cells (FIG. 11). These data show that IL-21 plays a key role in promoting accrual of Tfh10 cells with age, whose production of IL-10 likely feeds back to suppress IL-6.

### IL-21 Promotes Repression of Bim in Aged Tfh10 Cells Leading to Their Enhanced Survival

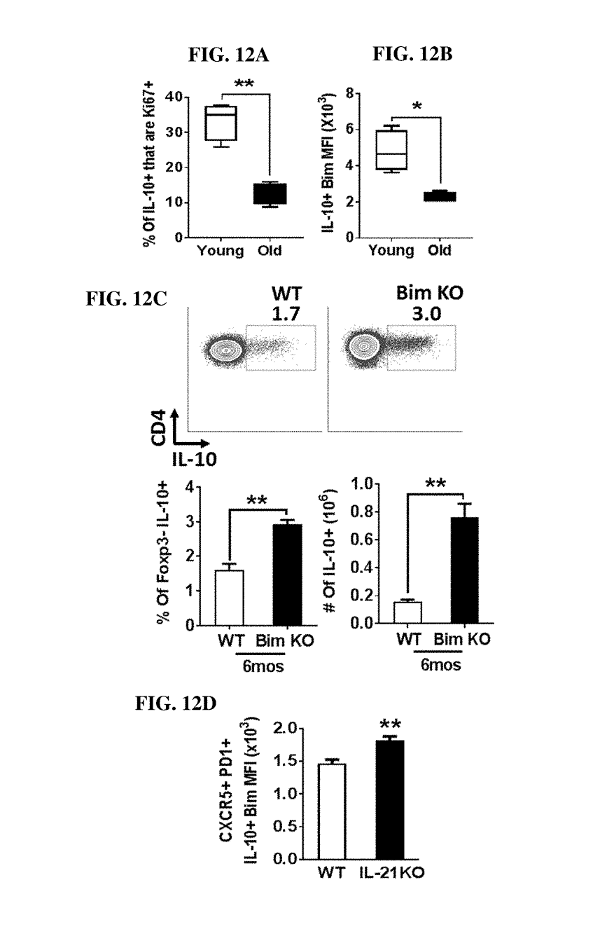

The accumulation of Tfh10 cells with age could be due to their increased proliferation and/or increased survival. The frequency of Tfh10 T cells that stained positive for the proliferation marker Ki-67+ actually decreased with age, ruling out the possibility that increased proliferation explains their accrual (FIG. 12A). Given our and others previous data implicating the pro-apoptotic molecule Bim in T cell survival, (Chougnet, C. et al., J. Immunol. 186:156-163 (2011); Tsukamoto, H. et al., J. Immunol. 185: 4535-4544 (2010)), we examined the role of Bim in the survival of IL-10-producing CD4+ T cells. First, Bim levels were reduced in IL-10-producing CD4+ T cells from aged compared to young mice (FIG. 12B). Second, the frequency and total number of FoxP3− CD4+ T cells that were IL-10+ was significantly increased in Bim-deficient mice, as early as 6 months of age (FIG. 12C). Given that IL-21 promotes accumulation of Tfh10 cells, we next determined whether IL-21 contributed to their reduced expression of Bim. Indeed, IL-21 was critical to suppress the levels of Bim within Tfh10 cells, which likely contributes to their increased survival (FIG. 12D). Together, these data suggest that IL-21-driven suppression of Bim contributes to the accumulation of IL-10-producing cells by enhancing their survival.

### Tfh10 Cells in Aged Mice Manifest Diminished Levels of BCL6 Thereby Enabling IL-10 Expression

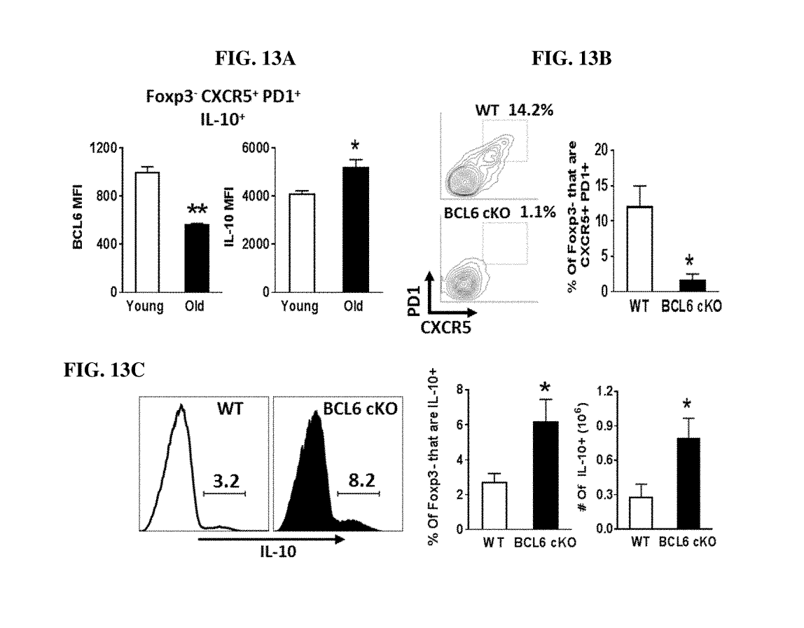

BCL6 is essential for Tfh differentiation and is induced by IL-6 and IL-21, so we examined BCL6 levels in young versus aged Tfh cells. Interestingly, BCL6 levels were actually decreased in aged Tfh10 cells (FIG. 13A). Further, decreased levels of BCL6 were associated with higher levels of IL-10 (FIG. 13A). To determine whether BCL6 is required for the accrual of Tfh cells as well as their production of IL-10 in aged mice we utilized CD4Cre-BCL6^{f/f} mice that have a T cell-specific loss of BCL6 (Hollister, K. et al., J. Immunol. 191:3705-3711 (2013)). As expected, given that BCL6 is critical for promoting Tfh cells (Yu, D. et al., Immunity 31:457-468 (2009)), CD4Cre-BCL6^{f/f} mice had a significant loss of CXCR5 and PD1 expressing Foxp3− CD4+ T cells (FIG. 13B). Strikingly, already by one year of age, the loss of BCL6 led to a significant increase in the frequency and total number of CD4+ FoxP3− cells that produced IL-10 (FIG. 13C). As BCL6 has been reported to suppress expression of Blimp1 and Blimp1 has been shown to promote IL-10 expression from T cells (Neumann, C. et al., J. Exp. Med. 211:1807-1819 (2014)), we next examined Blimp1 levels in aged mice with and without BCL6. Interestingly, the levels of Blimp1 did not change in Tfh cells from aged mice, whether or not BCL6 was present, making it unlikely that Blimp1 is promoting increased IL-10 expression in aged Tfh cells (data not shown). Thus, BCL6 is critical for suppressing IL10-producing CD4+ FoxP3− T cells.

### IL-10 Limits Tfh-Dependent Vaccine Responses in Aged Mice.

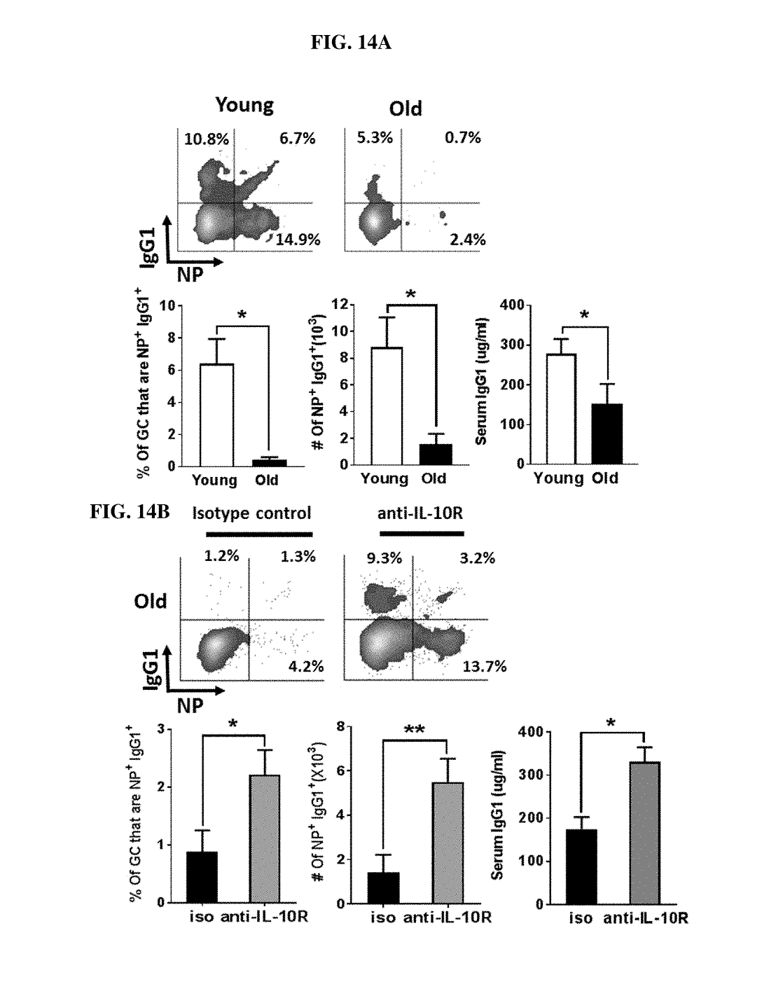

We next sought to determine physiologic relevance of Tfh10 cells with age. As vaccine responsiveness is a major problem in elderly humans we used a classic mouse model of a Tfh-dependent antibody response, immunization with NP-KLH. We reasoned that, if Tfh10 cells were important for regulating vaccine responses, then limiting IL-10 signaling should affect vaccine responsiveness. Old mice displayed a significantly lower level of anti-NP antibody production, as well as significantly lower frequency and total numbers of NP-specific B cells compared to young mice (FIG. 14A). Neutralization of IL-10R during NP-KLH immunization significantly restored anti-NP antibody production as well as the frequency and numbers of anti-NP-specific B cells to levels close to those observed in young mice (FIG. 14B). Thus, IL-10 limits Tfh-dependent B cell responses in aged mice.

### Tfh10 Cells Accumulate During Aging in Humans.

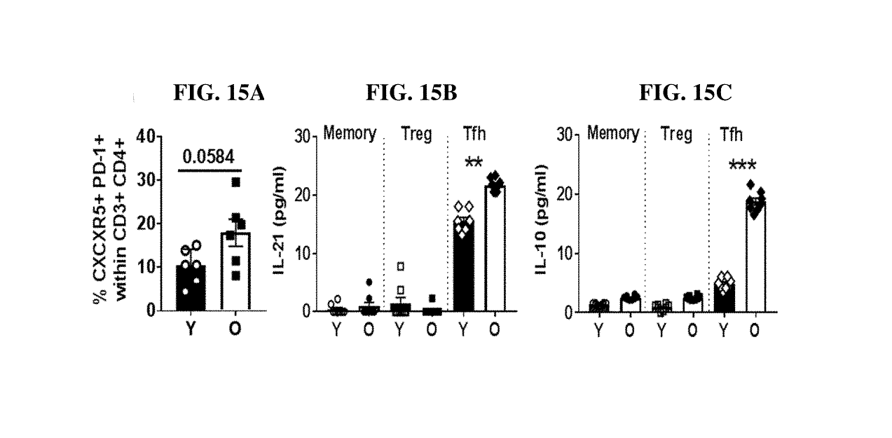

Given the above data in mice implicating Tfh10 cells as regulators of vaccine responsiveness, we next determined whether Tfh10 cells accumulated in aged humans. As Tfh cells are mainly located, and function, in secondary lymphoid organs, we analyzed their proportion in the spleens of young and old organ donors with no immunologic condition. Importantly, the frequency of Tfh cells (CXCR5+PD-1+) was increased in aged humans (FIG. 15A). Because flow cytometric analysis of cytokines is affected by cryopreservation, we FACS-sorted memory CD4+ T cells (CD45RO+) into Tfh (CD25−CD127+PD-1+CXCR5+), Treg (CD25+CD127−PD-1−CXCR5−) and other memory cells (CD25−CD127+PD-1−CXCR5−) and analyzed their production of IL-10 and IL-21 after in vitro re-stimulation with anti-CD3/CD28 beads. As expected, IL-21 production was largely limited to Tfh cells and was increased with age (FIG. 15B). Strikingly, the population with the highest production of IL-10 was the old Tfh cells (FIG. 15C). Thus, similar to mice, Tfh10 cells accumulate in aged humans and may explain their well-known age-related impairment in vaccine responsiveness.

Our data demonstrate a connection between two age-related phenomena, chronic inflammation (“inflammaging”) and immune suppression. Notably, we found that IL-6 (a hallmark of inflammaging) is critical for the emergence of IL-10-expressing Tfh cells in aged mice and that IL-10 provides a negative feedback mechanism to dampen IL-6 driven inflammaging. However, our data also show that the increase in systemic IL-10 impairs vaccine responsiveness. While cell intrinsic defects in adaptive immune cells are known to contribute to age-related immune suppression, our data also show a substantial contribution of IL-10 to age-related immune suppression. Our data also indicate that cell intrinsic defects can at least partially be reversed by inhibition of IL-10.

In our aged cohorts of mice, neither Tfr nor Treg appear to be substantial contributors to IL-10 production in vivo. Rather, our data show that IL-10-producing Tfh cells are required for elevated systemic levels of IL-10 in aged mice.

We also show that IL-6 and IL-21 are individually required to drive the accumulation of Tfh10 cells. Our data indicate that IL-6 is necessary for the development, but not maintenance, of Tfh10 cells. In contrast, our data support a requirement for IL-21 in the long-term maintenance of Tfh10 cells. Our data also provide some molecular insight into the role of IL-21 on maintenance of Tfh10 cells in that we show it is required to suppress their expression of Bim, which regulates their long-term survival.

Our data also show that loss of BCL6, a transcription factor essential for Tfh development, enhanced IL-10-producing CD4+ FoxP3− T cells. Although we were unable to ascribe a Tfh status to these cells as the signature markers CXCR5 and PD1 were both substantially reduced in the absence of BCL6, our data clearly show that BCL6 is a major negative regulator of IL-10 production. This result likely reveals the dual nature of BCL6. On the one hand, BCL6 is critical for Tfh development and for expression of the canonical Tfh markers CXCR5 and PD1, while on the other hand, BCL6 represses IL-10 expression. Indeed, we find little IL-10-production from Tfh cells in young mice, who maintain high expression of BCL6. However, with age, BCL6 levels decline and IL-10 production from Tfh cells increases.

Repression of IL-10 by BCL6 in Tfh10 cells could occur via two, non-mutually exclusive mechanisms, either directly by binding to sites in the IL-10 locus or indirectly, through repression of Prdm1 (Blimp1) expression since Blimp1 promotes IL-10 expression in both CD4+ and CD8+ T cells (Neumann, C. et al., J. Exp. Med. 211:1807-1819 (2014)). However, we recently showed that the additional loss of Blimp1 did not reduce enhanced IL-10 production in the absence of BCL6 in young mice (Xie, M. et al., Eur. J. Immunol. 47:1136-1141 (2017)). Further, while BCL6 levels were reduced in aged T cells, Blimp levels were not increased substantially in aged T cells whether or not BCL6 was present (data not shown). Thus, our data do not support a significant role for Blimp1 in controlling IL-10 production from Tfh10 cells.

In summary, our data show that Tfh cells are a major source of T cell-derived IL-10 and that blockade of IL-10 signaling largely restores vaccine responsiveness in aged animals. These data indicate that blockade of IL-10 can enhance vaccine responsiveness in at-risk populations such as the elderly. A transient inhibition of IL-10 in conjunction with vaccination would likely avoid any detrimental effects that might otherwise result from long-term inhibition of this anti-inflammatory cytokine.

### Materials and Methods

### Mice

Young (6-weeks to 4-months) C57BL/6 mice were purchased from Taconic Farms (Germantown, N.Y.). Old (≥16 months) or Middle age (12-15 months) C57BL/6 mice were from National Institutes of Aging colony located at Charles River Laboratories (Wilmington, Mass.). Foxp3-IRES-DTR-GFP knock-in C57BL/6 mice (Kim, J. et al., Nat. Immunol. 8:191-197 (2007)), were a generous gift from Dr. A. Rudensky and were aged in house. Bim-deficient (Bim knockout) mice were originally a kind gift from Drs. P. Bouillet and A. Strasser and were bred in-house. IL-6-deficient (IL-6 KO) mice on the C57BL/6 background were aged in-house. IL-10-reporter (VertX) mice which possess an IL-10-IRES-eGFP cassette in the endogenous IL-10 locus on the C57BL/6 background (Madan, R. et al., J. Immunol. 183:2312-2320 (2009)), were aged in-house. Young, middle age and old Germ-free mice on the C57BL/6 background were maintained in isolator units in the CCHMC Gnotobiotic Mouse Facility. Young and old FoxP3-fate mapping mice (Foxp3^{Crc}Rosa26^{dTomato}) on the C57BL/6 background were kindly provided by Dr. Sing S. Way (CCHMC). IL-17A fate tracking mice IL-17^{Cre}Rosa26e^{YFP} (Hirota, K. et al., Nat. Immunol. 12:255-263 (2011)) on the C57BL/6 background were bred and aged under specific-pathogen free conditions in the animal facility of the Research Centre Borstel, Germany. Young and old IL-21-deficient (IL-21KO) mice on the C57BL/6 background were bred, maintained and aged in fully accredited facilities at the University of Alabama at Birmingham. Spleens (controls and IL-21KO) were shipped overnight on ice and analyzed in Cincinnati. CD4^{Cre}BCL6^{f/f} mice on the C57BL/6 background were bred, maintained and aged in fully accredited facilities at the University of Indiana. Spleens (CD4^{Cre}BCL6^{f/f} and control) were shipped overnight on ice and analyzed in Cincinnati. All animal protocols were reviewed and approved by the Institutional Animal Care and Use Committee at the Cincinnati Children's Hospital Research Foundation (IACUC 2016-0087).

### Immunization, Neutralization and Depletion Treatments

For depletion of Foxp3 Treg cells in old FoxP3-DTR mice, 1.25 μg DT/mouse was intraperitoneally injected and were sacrificed two days later. For CD4 T cell depletion, mice were injected with a single dose of 600 μg/mouse of anti-CD4 intraperitoneally (Clone: YTS191 BioXcell) or isotype control (Clone: LFT-2 BioXcell) and were sacrificed two days later. For T cell-dependent immunization, mice were immunized intraperitoneally with 100 μg NP-KLH (Biosearch Technologies) mixed with 50% (vol/vol) alum (Thermo Scientific) and sacrificed 20 days later. For IL-10R neutralization, mice were injected with anti-IL-10R blocking antibody (Clone: 1B1.3A BioXcell) or ratIgG1 isotype control (Clone: HRPN BioXcell) at day −1 (1 mg), day 1 (250 μg), day 3 (500 μg), day 6 (500 μg), day 8 (250 μg) and were sacrificed 10 days after immunization. For IL-6 neutralization mice were injected intraperitoneally with 300 μg α-IL-6 (Clone: MP5-20F3, BioXcell) or 300 μg isotype control (Clone: HRPN BioXcell) on days 0 and sacrificed on day 2. For ICOS-L neutralization old C57BL/6 mice were injected intraperitoneally with 150 μg anti-ICOSL (HK5.3, BioXcell) or with rat IgG2A isotype control (2A3, BioXcell), on days 0, 3, 6, 9 and sacrificed on day 12.

### In Vivo Cytokine Capture Assay and ELISAs

IL-6 and IL-10 in vivo cytokine capture assay was performed as previously described (Finkelman, F. et al., Curr. Prot. Immunol., Ch. 6, Unit 28 (2003)) employing biotinylated capture antibodies (Invitrogen). In brief, young (1.5-4 months) and old (≥16 mo) C57BL/6 mice were injected i.v. with 10 μg biotinylated anti-IL-6 (MP5-32C11; Invitrogen) and anti-IL-10 (JESS-16E3: Invitrogen)) capture antibodies; mice were bled within 24 h and serum was collected. A luminescent ELISA was performed using anti-IL-6 (MP5-20F3; Invitrogen) or anti-IL-10 (JESS-2A5: BD Biosciences) as the coating antibody. For NP-specific antibody titers, 96-well plates were coated overnight at 4° C. with NP30-BSA (Biosearch), followed by blockade of nonspecific biding by incubation for 1-2 h at 25° C. with 5% BSA. Serum samples were loaded into plates with eight serial dilutions (starting from 1:100 or 1:1000), followed by incubation for 2 h at 25° C. or overnight at 4° C. After samples were washed, horseradish peroxidase (HRP)-conjugated goat antibody to mouse IgG1 (PA1-74421; Thermo) was added to plates, followed by incubation for 2 hr at 25° C. The reactions were developed by incubation for 15 min at 37° C. with 50 μl TMB substrate (BioLegend) and were stopped by the addition of 25 μl 10% H₃PO₄. The plates were read at 450 nm and 570 nm (for correction) with an enzyme-linked immunosorbent assay reader.

### RT-PCR

Samples from different tissues were homogenized and total cellular RNA was extracted and quantified. DNase-treated RNA was then used to synthesize cDNA. The primer sequences used for detection of IL-10 were: 5′-GCTCTTACTGACTGGCATGAG-3′ and 5′-CGCAGCTCTAGGAGCATGTG-3′. Expression levels were normalized to S14 as internal control gene. The primer sequences used for S14 detection were 5′-GAG TCT GGA GAC GA-3′ and 5′-TGG CAG ACA CCA AAC ACA TT-3′. Quantitative real-time PCR was performed with Roche LightCycler 480 SYBRGreen 1 Master Mix using the Roche LightCycler 480 II instrument (Roche Diagnostics). Each reaction was performed in triplicate.

### Flow Cytometry and Cell Sorting: Human Studies

Spleen cells from young (median: 18.8, range 18-26 yrs, 3 males, 5 females) and old (median: 62, range 60-67 yrs, 4 males, 4 females) organ donors with no immunological condition were rested overnight in RPMI medium supplemented with 10% fetal calf serum (FCS), 1% penicillin, streptomycin, glutamine and 0.5% HEPES, at 37° C. and 5% CO2. The cells were then washed with PBS+2% FCS and stained for CD4, CXCR5, PD-1, CD45RO (Biolegend), CD25 (BD Bioscience), CD3 (Invitrogen), and CD127 (Beckman Coulter) for 30 min. in 4° C., fixed with 4% para-formaldehyde for 20 mins in 4° C. Cells were stained for Foxp3 (Invitrogen) using Invitrogen Foxp3 permeabilization buffer and acquired on a flow cytometer. For sorting, CD4+ T cells were bead-purified by negative selection from spleen cells, surface stained with Abs against CD45RO, CD127, CD25, PD-1, CXCR5 and the following populations were sorted by FACS after gating on memory CD4+ T cells (CD45RO+): Tfh (CD25−CD127+PD-1+CXCR5+), Treg (CD25+CD127−PD-1−CXCR5−) and non Tfh memory cells (CD25−CD127+PD-1−CXCR5−). 10,000 cells were stimulated in vitro with anti-CD3/CD28 beads at a 1:1 cell: bead ratio, or unstimulated. After 16 hr, supernatants were collected and analyzed by Luminex.

### Flow Cytometry and Cell Sorting: Mouse Studies

Spleens were harvested and crushed through 100-mm filters (BD Falcon) to generate single-cell suspensions. A total of 2×10⁶ cells plated incubated with Fc block and were surface stained with a combination of the following Abs for surface staining: −CD4, −CD8α, −TCRβ, −LAG3, −Fas (BD Biosciences), −CD19, −PD1, −CXCR5, −GL-7, CD49B (Invitrogen), B220, IgG1(Biolegend). Cells were intracellularly stained with antibodies against Bim (Cell Signaling Technology), Ki67, Foxp3 (Invitrogen), BCL6 (BD Biosciences). For cytokine staining, cells were stimulated with 25 ng/ml PMA and 0.5 m/ml ionomycin for 5 hours, in the presence of brefeldinA for the final 4 h and fixed with 2% methanol-free formaldehyde for 1 hour followed by intracellular staining for IL-10, IFN-γ (Biolegend), IL-17, IL-4 (Invitrogen) using Invitrogen Foxp3 permeabilization buffer. For IL-21 staining, cells were fixed, permeabilized with perm buffer from Invitrogen and incubated with IL-21R/Fc (R&D systems) chimera for 45 min.-1 hr at 4° C. Cells were then washed with perm buffer and stained with AF488 or AF-647-conjugated affinity-purified F(ab′)2 fragment of goat anti-human Fcγ antibody (Jackson ImmunoResearch Laboratories) for 45-1 hr at 4° C. Data were acquired on an LSRII flow cytometer (BD Biosciences) and analyzed using FACSDiva software (BD Biosciences) or FlowJo software (FlowJo, Ashland, Oreg.). For sorting, spleen cells from young (3 mo, n=3) and old (>18 mo, n=4) Foxp3-IRES-DTR-GFP mice were enriched for CD4+ T cells using the negative selection MACS CD4+ T cell isolation kit II (Miltenyi Biotec, San Diego, Calif.). Enriched cells were stained with anti-CD4, -CD44, -CD62L Abs, and the following populations were sorted by a FACSAria (BD Biosciences): CD4+ Foxp3GFP+ (Treg), CD4+ Foxp3-GFP− CD44^{lo} CD62L^{hi} (naive CD4+), CD4+ Foxp3-GFP− CD44^{hi} CD62L^{lo} (memory CD4+). All three populations were stimulated with 50 ng/ml PMA and 1 m/ml ionomycin and supernatants were collected after 15 hours.

### Single Cell RNA-Sequencing

Young and old CD4+GFP+ CD25−CD69− cells were sorted and post sort analysis showed that <1% of the sorted cells were FoxP3+. Single cells were isolated on a FLUIDIGM C1 instrument, cDNA isolated, amplified, barcoded, and subjected to high throughput sequencing. Following transcript pseudoalignment and normalization using Kallisto in AltAnalyze, predominant gene expression identified from individual cells by ICGS. ICGS provides the sensitivity to detect rare and common cell populations as well as possible transitional cell states. Four major cell populations were reported along with statistically enriched Gene Ontology terms for gene clusters.

### EQUIVALENTS

Those skilled in the art will recognize or be able to ascertain using no more than routine experimentation, many equivalents to the specific embodiments of the invention as described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A method for immunizing a subject in need thereof with a prophylactic vaccine, the method comprising enhancing the subject's immune responsiveness to the vaccine by administering an IL-10 inhibitor to the subject.

2. The method of claim 1, wherein the subject in need is an elderly human, a human who has received one or more immunosuppressive agents as part of a therapeutic regimen, for example a chemotherapy regimen or a regimen to prevent rejection in a solid organ transplant recipient, a human who has received one or more regimens of radiation therapy, a human stem cell transplant recipient, a subject having graft-versus-host disease, a subject having HIV, a subject having end stage renal disease, a subject having end stage diabetes, and a subject having end stage cirrhosis.

3. The method of claim 2, wherein the subject in need is an elderly human, preferably at least 50 years of age, most preferably at least 65 years of age.

4. The method of claim 1, wherein the IL-10 inhibitor is an agent that inhibits the binding of IL-10 to its receptor, IL-10R.

5. The method of claim 4, wherein the agent is a peptide, a polypeptide, a small organic molecule, or an antibody

6. The method of claim 5, wherein the IL-10 inhibitor is a monoclonal antibody, preferably a human or humanized monoclonal antibody.

7. The method of claim 1, wherein the IL-10 inhibitor is an agent that transiently inhibits the survival of follicular helper T (“Tfh”) cells.

8. The method of claim 1, wherein the IL-10 inhibitor is an agent that inhibits IL-10 production by follicular helper T (“Tfh”) cells.

9. The method of claim 8, wherein the inhibitor is a single or double stranded RNA interference-based agent (RNAi) targeted to inhibit the expression of the IL-10 gene or the IL-10 receptor gene.

10. The method of claim 9, wherein the inhibitor is selected from a microRNA, a short hairpin RNA, or a short interfering RNA (siRNA).

11. The method of claim 10, wherein the inhibitor is an siRNA, optionally conjugated to a targeting moiety via an optional linker.

12. The method of claim 11, wherein the targeting moiety targets delivery of the siRNA to Tfh cells.

13. The method of claim 12, wherein the targeting moiety comprises a CXCR5 ligand.

14. The method of claim 13, wherein the CXCR5 ligand is the chemokine (C-X-C motif) ligand 13 (“CXCL13”), or a CXCR5 binding fragment thereof.

15. The method of claim 1, wherein the method further comprises administering the prophylactic vaccine to the subject in need thereof.

16. The method of claim 1, wherein the prophylactic vaccine is selected from a vaccine against influenza, Streptococcus pneumoniae, tetanus, diphtheria, pertussis, respiratory syncytial virus (RSV), typhoid fever, Japanese encephalitis, yellow fever, Hepatitis A and Hepatitis B.

17. The method of claim 16, wherein the prophylactic vaccine is an influenza vaccine.

18. The method of claim 1, wherein the prophylactic vaccine is a Tfh-dependent vaccine.

19. The method of claim 1, wherein the IL-10 inhibitor is administered before, concurrently with, or after the administration of the prophylactic vaccine.

20. The method of claim 19, wherein the IL-10 inhibitor is administered substantially at the same time as the prophylactic vaccine.

21. The method claim 1, wherein the subject in need does not have a chronic infection.

22. The method of claim 1, wherein the Tfh cells are defined by the positive cell surface expression of the CD4, CXCR5, and PD1 marker proteins in the absence of FoxP3 expression, i.e., FoxP3⁻ CD4+ CXCR5+ PDF1⁺.

23. The method of claim 1, wherein the IL-10 inhibitor is encapsulated in a liposome-based nanoparticle comprising a targeting moiety selected from an anti-CXCR5 antibody and CXCL13, or an IL-10 receptor binding fragment thereof.

24. (canceled)


SEQUENCE LISTING (4 sequences)

SEQ ID NO: 1 - DNA, 21 bp, Homo sapiens
gctcttactgactggcatgag

SEQ ID NO: 2 - DNA, 20 bp, Homo sapiens
cgcagctctaggagcatgtg

SEQ ID NO: 3 - DNA, 14 bp, Homo sapiens
gagtctggagacga

SEQ ID NO: 4 - DNA, 20 bp, Homo sapiens
tggcagacaccaaacacatt